(19) United States
(12) Patent Application Publication
(10) Pub. No.: US 2023/0116387 A1
(43) Pub. Date: Apr. 13, 2023

(54) HIGH-PURITY PURIFICATION TECHNIQUE FOR Gc PROTEIN

(71) Applicant: KABUSHIKI KAISHA MEDICALBIARA, Tokyo (JP)

(72) Inventors: Sachiko TAKAHASHI, Tokyo (JP); Masumi UEDA, Tokyo (JP); Yuki MORITA, Tokyo (JP); Masaru TANOKURA, Tokyo (JP); Takuya MIYAKAWA, Tokyo (JP); Xuyang LI, Tokyo (JP); Tomonari MURAMATSU, Tokyo (JP); Rong WANG, Tokyo (JP)

(73) Assignee: KABUSHIKI KAISHA MEDICALBIARA, Tokyo (JP)

(21) Appl. No.: 17/907,144

(22) PCT Filed: Mar. 3, 2021
(86) PCT No.: PCT/JP2021/008050

§ 371 Date: Sep. 23, 2022

(30) Foreign Application Priority Data
Mar. 25, 2020 (JP) 2020-055080

(51) Int. Cl. C07K 1/22; C12P 21/00; C07K 1/18; C07K 14/47

(52) U.S. Cl. CPC C07K 1/22; C07K 1/18; C07K 14/47; C12P 21/00

(57) ABSTRACT

An improved method for purifying Gc protein to a high purity, and a method for producing GcMAF are provided.

According to the present disclosure, Gc protein is purified through a combination of affinity chromatography and anion exchange chromatography, and thus, Gc protein can be purified to a higher purity as compared with a conventional purification method using affinity chromatography alone. As a result, GcMAF can be efficiently produced.

### CROSS REFERENCE TO RELATED APPLICATION(S

This is the National Stage of International Application No. PCT/JP 2021/008050, filed Mar. 03, 2021, which claims the benefit of Japanese Patent Application No. 2020-055080 filed Mar. 25, 2020, and the disclosure of which are hereby incorporated by reference in their entirety.

### TECHNICAL FIELD

The present disclosure relates to an improved method for purifying Gc protein and a novel method for producing GcM AF using the relevant method.

### BACKGROUND ART

Cancer is now one of the most common causes of death in developed countries. In recent years, various cancer treatment methods including chemotherapy, radiation therapy and biological preparations have been developed, but the rate of deaths due to cancer is still increasing. Under such circumstances, cancer immunotherapy is attracting attention as one of approaches to cancer treatment through activation of the immune system such as T cells, dendritic cells, natural killer cells, or macrophages.

In activation of macrophages, B lymphocytes and T lymphocytes as well as serum vitamin D binding protein need to be involved.

Serum vitamin D binding protein has a relative molecular weight of about 52,000 and is one of the glycoproteins present in serum. The protein is known to have a function to mediate transport of vitamin D and hydrolysis metabolites thereof. Serum vitamin D binding protein is also referred to as Gc protein, and is known to function as a precursor of a Gc protein-derived macrophage activating factor (hereinafter referred to as “GcMAF”).

Gc protein has an O-linked type sugar structure in which N-acetylgalactosamine (GalNac) is covalently bound to Thr418 or Thr420, and galactose and sialic acid or mannose are linked to each other and are converted to GcMAF through sugar chain cleavage caused by β-galactosidase of stimulated B lymphocytes and sialidase secreted from T lymphocytes. After the enzyme action, GcMAF has GalNac only.

As a method for purifying Gc protein from human blood, Link et al. first reported a purification method by affinity chromatography using a 25-hydroxyvitamin D3-immobilized column (Link et al., Analytical Biochemistry 157, 262-269, 1986), and thereafter, Yamamoto et al. established a method for enzymatically converting Gc protein to GcMAF (Yamamoto et al., Translational Oncology 1, 65-72, 2008).

Extrinsic GcMAF thus prepared has been applied to various assays. For example, it has been reported that GcMAF directly inhibits growth of human prostate and breast cancer cells (Gregory et al., PLos ONE 5, e13428, 2010; and in Pacini et al., Anticancer Research 32, 45-52, 2012), and also in in vitro experiments using some model mice, the inhibitory effect of GcMAF on tumor growth and angiogenesis has been described (Toyohara et al., Oncology Letters 2, 685-691, 2011; Nonaka et al., Journal of Surgical Research 172, 116-122, 2012; Yamamoto et al., Cancer Research 57, 2187-2192, 1997; and Kisker et al., Neoplasia 5, 32-40, 1997). In addition to these reports, some research groups have suggested applicability of extrinsic GcMAF to cancer immunotherapy (Inui et al., Anticancer Research 34, 4589-4593, 2014; Ruggiero et al., Anticancer Research 34, 3569-3578, 2014; Thyer et al., Oncoimmunology 2, e25769, 2013; Inui et al., Anticancer Research 33, 2917-2919, 2013; and Ward et al., American Journal of Immunology 10, 23-32, 2014).

For application of GcMAF to the above medical uses and development as a therapeutic agent, GcMAF as well as Gc protein serving as a precursor of GcMAF are demanded to be provided at a high purity.

When the present inventors additionally examined the purification method described in the literature of Link et al., however, it was found that a satisfactory level of purification purity of Gc protein cannot be achieved yet by this method as described in the examples below.

### SUMMARY OF DISCLOSURE

### Technical Problem

Accordingly, it is an object of the present disclosure to provide an improved method for highly purifying Gc protein, and a method for producing GcMAF.

### Solution to Problem

The present inventors made earnest studies to solve the above problems, and, as a result, found that Gc protein can be purified to a high purity by performing purification by a combination of affinity chromatography and anion exchange chromatography as compared with the conventional purification method using affinity chromatography alone, and furthermore found that GcMAF can be thus efficiently produced, resulting in the present disclosure.

Therefore, the present disclosure provides the following:
[1] A method for purifying Gc protein, comprising the steps of: (a) purification by affinity chromatography; and (b) purification by anion exchange chromatography.
[2] The method according to [1], wherein the step (b) of purification by anion exchange chromatography is performed after the step (a) of purification by affinity chromatography.
[3] The method according to [1] or [2], wherein a vitamin D3-immobilized affinity column is used in the step (a) of purification by affinity chromatography.
[4] The method according to any one of [1] to [3], wherein a buffer having a salt concentration of more than 150 mM to 2 M is used as a buffer for washing the protein not bound to a column in the step (a) of purification by affinity chromatography.
[5] The method according to any one of [1] to [4], wherein a surfactant and/or a chelating agent is not used in the step (a) of purification by affinity chromatography.
[6] The method according to any one of [1] to [5], wherein subsequently to the step (a) of purification by affinity chromatography, a column eluate resulting from the step (a) is dialyzed against a buffer of pH to 10.
[7] The method according to any one of [1] to [6], wherein a resin column having a cationic functional group selected from the group consisting of quaternary ammonium, diethyl aminoethyl, aminoethyl, para-aminobenzyl, and guanide ethyl is used in the step (b) of purification by anion exchange chromatography.
[8] The method according to any one of [1] to [7], wherein the Gc protein is eluted with the concentration gradient of a salt concentration of 0 to 1 M in the step (b) of purification by anion exchange chromatography.
[9] The method according to any one of [1] to [8], wherein the Gc protein is purified from human plasma or human serum.
[10] A method for producing a Gc protein-derived macrophage activating factor (GcMAF), comprising the steps of: (i) purification of Gc protein according to [1]; and subsequently (ii) conversion to GcMAF by contacting the Gc protein with an enzyme.
[11] The method according to [10], wherein β-galactosidase is used as the enzyme.

### Effects of Disclosure

According to the present disclosure, Gc protein can be highly purified, and thus, GcMAF can be efficiently produced. In addition, from the thus purified Gc protein, GcMAF that has immunoregulatory activity which may inhibit tumor growth and angiogenesis and is expected to be applied to various medical uses can be efficiently produced.

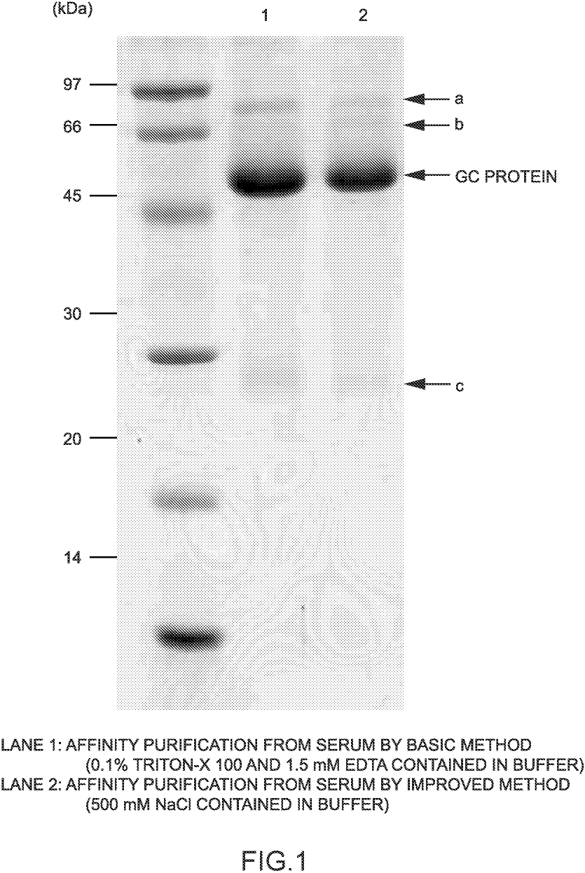

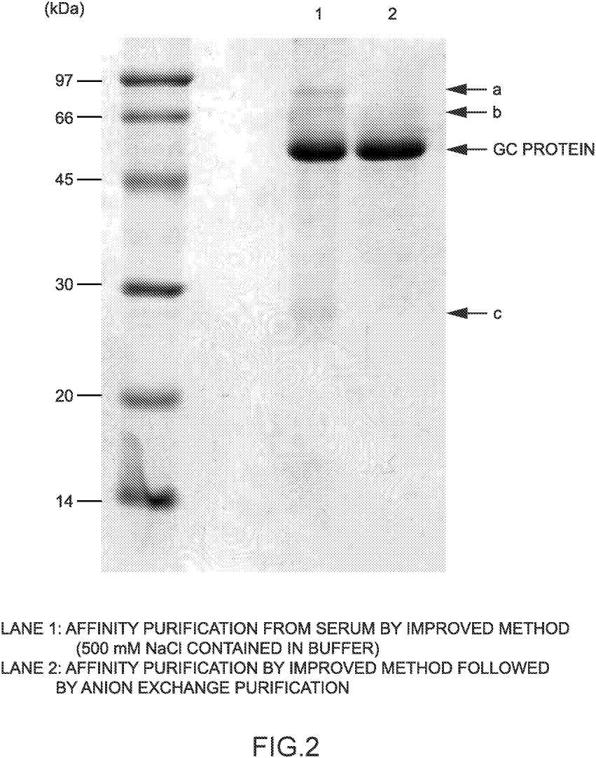

### BRIEF DESCRIPTION OF DRAWINGS

[FIG. 1] Comparison of protein purification patterns obtained after purification by affinity chromatography (25-OH-D3-immobilized column chromatography). Respective lanes indicate the following: M: molecular weight marker, lane 1: purification pattern obtained by a basic method, and lane 2: purification pattern obtained by an improved method, a, b, and c respectively indicate bands of contaminated proteins visualized with CBB dyeing.

[FIG. 2] Comparison of protein purification patterns between before and after purification by anion exchange chromatography (RESOURCE Q column chromatography). Respective lanes indicate the following samples. M: molecular weight marker, lane 1: protein purification pattern obtained after affinity chromatography by the improved method, lane 2: protein purification pattern of a fraction containing Gc protein obtained after purification by anion exchange chromatography subsequently performed after the affinity chromatography by the improved method. a, b, and c respectively indicate bands of contaminated proteins visualized with CBB dyeing.

### DESCRIPTION OF EMBODIMENTS

The present disclosure relates to a method for purifying Gc protein by a combination of affinity chromatography and anion exchange chromatography, and a method for producing GcMAF.

### Gc Protein

In the present disclosure, “Gc protein” encompasses glycoproteins, including subtypes of Gc1, Gc2, Gc1f, Gc1s, and the like having an O-linked type sugar structure in which N-acetylgalactosamine (GalNac) is covalently bound to Thr418 or Thr420, and galactose and sialic acid or mannose are linked to each other, genetic variants thereof, and all of the related various forms including biological active variants thereof, and activated fragments thereof.

Gc protein to be purified in the present disclosure may be in a form contained in a human-derived or nonhuman animal-derived biological homogenate, body fluid, blood or the like, or in a form of a crude purified product obtained by another known method.

In a specific embodiment, human blood, preferably human plasma or human serum, is used as a sample containing Gc protein because thus Gc protein can be purified efficiently and in a large amount.

### GcMAF

In the present disclosure, “GcMAF” relates to Gc protein having an amino acid residue, typically a N-acetylgalactosamine group linked to Thr, and is capable of activating a macrophage, or an activated fragment thereof, and here, the “activated fragment” includes any portion of Gc protein having an amino acid residue, typically a N-acetylgalactosamine group linked to Thr, and is capable of activating a macrophage.

In a specific embodiment, “GcMAF” means Gc protein having, as a sugar chain, only a N-acetylgalactosamine group linked to Thr418 or Thr420, and is capable of activating a macrophage.

### Affinity Chromatography

Purification by affinity chromatography of the present disclosure is performed in accordance with procedures of the method of Link et al. described above. Preferably, a sample containing Gc protein is caused to pass through an affinity column such as an actin-immobilized column, a vitamin D-immobilized column, or an anti-Gc globulin antibody-immobilized column for causing the Gc protein to specifically bind to the column, a contaminated protein not binding to the column is removed with an appropriate washing solution, and ultimately, the Gc protein is eluted with an appropriate eluate.

In a specific embodiment, a vitamin D3-immobilized column, and preferably 25-hydroxy-vitamin D3-immobilized column is used as the affinity column. Here, 25-hydroxy-vitamin D3 is vitamin D3 hydrated at position 25, is abbreviated also as 25-OH-D3, and is known to have very strong specific binding force to Gc protein.

A solid support of the affinity column can be, but is not limited to, any of the various materials known to be usable as the solid support, such as cellulose, a cellulose derivative, sepharose, silica, a hydrophilic vinyl polymer, a metal, glass, ceramics, and a resin. Sepharose, silica, or a hydrophilic vinyl polymer is preferably used, and sepharose or a hydrophilic vinyl polymer is particularly preferably used. A commercially available solid support can also be used, and, for example, Sepharose CL-6B resin (GE Healthcare Limited) or TOYOPEARL resin (Tosoh Corporation) is used.

In a specific embodiment, a bond between vitamin D3 and the solid support can be caused through a covalent bond or a non-covalent bond, and the bond can be caused, for example, by a method in which vitamin D3 is epoxidized to be reacted with the solid support to which a thiol group has been introduced (Rebecca, P. et al., ANALYTICAL BIOCHEMISTRY, 157, 262-269, 1986), or a method in which vitamin D3 is aminopropyl etherified, a carboxyl group of the solid support is esterified with N-hydroxysuccinimide (NHS) to obtain an activated ester group, and the resultants are reacted with each other (Swamy, N. et al., PROTEIN EXPRESSION AND PURIFICATION, 6. 185-188 (1995)).

In a specific embodiment, a 25-OH-D3 sepharose column is used.

As a column washing solution, any of the buffers known to be usable for washing an affinity column can be used, and a buffer such as Tris is preferably used.

In the present disclosure, the buffer is preferably prepared to a rather high salt concentration, and can be prepared to a range of, for example, over 150 mM to 2 M, 200 mM to 1 M, or 350 mM to 700 mM. The buffer is particularly preferably prepared to a salt concentration of about 500 mM.

The type of the salt may be any of the salts known to be usable in a buffer, and for example, NaC1 or KC1 may be used, and NaCl is preferably used.

The pH of the buffer may be in a range of 5 to 9, 6 to 8, or 7 to 7.5, and is particularly preferably pH 7.4.

In a specific embodiment, a buffer containing 500 mM NaC1 and 20 mM Tris-HCl (pH 7.4) is used.

In the present disclosure, it is preferable to use neither a surfactant nor a chelating agent that must be removed in a subsequent treatment.

In a specific embodiment, it is preferable that a buffer containing a surfactant such as Triton X-100 and/or a chelating agent such as EDTA is not used.

The column may be pre-equilibrated with a buffer such as Tris before causing a sample containing Gc protein to pass therethrough.

In a specific embodiment, a buffer having the same composition as the column washing solution can be preferably used for equilibrating the column.

As a denaturing solution for eluting Gc protein from the column, a known denaturing solution such as an acetate buffer or a guanidine solution can be used.

In the present disclosure, a 1 M to 8 M guanidine hydrochloride solution can be preferably used, and a 6 M guanidine hydrochloride solution can be further preferably used.

Subsequently, a column eluate can be concentrated with an ultrafiltration membrane through known procedures.

The ultrafiltration membrane can be, but is not limited to, a commercially available concentration unit with an ultrafiltration membrane, such as Vivaspin 10,000 MWCO (GE Healthcare Limited).

In addition, the column eluate may be dialyzed through procedures known to those skilled in the art subsequent to or separate from the concentration with the ultrafiltration membrane.

As a dialysis solution, a known buffer such as Tris can be used. The pH of the dialysis solution may be in a range of 6 to 10, preferably 7 to 9, more preferably pH 7.5 to 8.5, and particularly preferably about 8.

In a specific embodiment, the dialysis is performed on 20 mM Tris-HCl (pH 8.0).

### Anion Exchange Chromatography

In the present disclosure, anion exchange chromatography may be performed through procedures known to those skilled in the art.

In a specific embodiment, purification by anion exchange chromatography is performed after the step of purification by the affinity chromatography described above in (3).

In the present disclosure, the anion exchange chromatography may be performed through known procedures, and preferably, a sample containing Gc protein (or the column eluate containing Gc protein if performed subsequently to the affinity chromatography) is caused to pass through a column using, as an anion exchanger, a resin having a cationic functional group such as quaternary ammonium, diethyl aminoethyl, aminoethyl, para-aminobenzyl, or guanide ethyl, and thus the Gc protein is eluted with a stepwise concentration gradient using a buffer having a prescribed salt concentration, or with a continuous concentration gradient. As the resin used in the column, a commercially available matrix containing, as a base, sepharose, dextran, acrylamide, silica, a hydrophilic vinyl polymer, ceramics or the like may be used.

In the present disclosure, DEAE cellulose, DEAE sepharose, QAE cellulose, or Q sepharose can be preferably used, and a commercially available anion-exchange column, such as Mini Chrom; TOYOPEARL(R) Giga Cap Q or Mini Chrom; TSKgel(R) Super Q (both Tosoh Corporation), RESOURCE(R) Q (GE Healthcare Limited), and Enricli(R) Q (Bio-Rad Laboratories, Inc.) can be used.

In a specific embodiment, RESOURCE(R) Q column (GE Healthcare Limited) is used.

The column may be pre-equilibrated with a known buffer such as Tris before causing a sample containing Gc protein (the column eluate if performed subsequently to the affinity chromatography) to pass therethrough.

The pH of the buffer may be in a range of 6 to 10, preferably in a range of 7 to 9, more preferably in a range of pH 7.5 to 8.5, and particularly preferably about 8.

In a specific embodiment, the column can be equilibrated with 20 mM Tris-HCl (pH 8.0).

As a buffer for eluting Gc protein, any of the known adequate buffers can be used without particular limitation, and a buffer such as Tris buffer can be used. In a specific aspect, a buffer that can minimize co-elution of contaminated proteins and has ionic strength for causing effective elution of Gc protein is preferably used.

The pH of the buffer may be in a range of 6 to 10, preferably in a range of 7 to 9, more preferably in a range of pH 7.5 to 8.5, and particularly preferably about 8.

In the present t disclosure, the elution of Gc protein may be performed continuously, or with a stepwise concentration gradient with multiple steps of prescribed salt concentrations.

In the present disclosure, the buffer is prepared to a salt concentration of preferably 0 to 2 M, and particularly preferably 0 to 1 M.

Regarding the type of the salt, a salt known to be usable in a buffer, such as NaCl or KC1 may be used, and NaCl is preferably used.

In a specific embodiment, the Gc protein is eluted with 20 mM Tris-HCl (pH 8.0) with a stepwise concentration gradient of 0 to 1 M NaCl.

Subsequently, the column eluate containing collected Gc protein fractions may be concentrated with an ultrafiltration membrane through known procedures.

The ultrafiltration membrane can be, but is not limited to, a commercially available concentration unit with an ultrafiltration membrane, such as Vivaspin 10,000 MWCO (GE Healthcare Limited).

### Production of GcMAF

In the present disclosure, GcMAF can be produced from GC protein purified as described above through known procedures, for example, by enzymatic conversion including stepwise removal of a specific oligosaccharide with a specific glycosidase enzyme, such as β-galactosidase.

In more detail, GcMAF can be produced through known procedures as follows:
(i) a step of mixing and reacting Gc protein purified by the method of the present disclosure with β-galactosidase bound to a solid support such as sepharose; and subsequently
(ii) a step of mixing and reacting the Gc protein having been treated with β-galactosidase further with sialidase or mannosidase.

As described above, Gc protein has an O-linked type sugar structure in which N-acetylgalactosamine (GalNac) is covalently bound to Thr418 or Thr420, and galactose and sialic acid or mannose are linked to each other.

Accordingly, in a specific embodiment, GcMAF containing only GalNac bound to Thr418 or Thr420 can be produced from Gc protein through stepwise sugar chain cleavage.

Hereinafter, examples of the present disclosure will be described, and it is noted that the present disclosure is not limited to these.

### EXAMPLES

### (Example 1) Collection of Blood

Fresh human blood collected with a medical grade blood collecting tube (Terumo Corporation) was centrifuged at 4,000 rpm for 15 minutes to separate the serum, and the resultant was stored at --80° until use.

### (Example 2) Purification of Gc Protein by Affinity Chromatography

### (i) Basic Method

Purification was performed by affinity chromatography in accordance with the method of Link et al. (Link et al., Analytical Biochemistry 157, 262-269, 1986). First, a column having 25-OH-D3 covalently bound thereto was equilibrated with a column buffer containing 50 mM Tris-HC1 (pH 7.4), 150 mM NaCl, 0.1% Triton X-100, and 1.5 mM EDTA, and thereafter, the human serum collected as described above was caused to pass through the column. Subsequently, the column was washed with a column buffer containing 50 mM Tris-HCl (pH 7.4) 150 mM NaC1, 0.1% Triton X-100, and 1.5 mM EDTA in 20-fold amount of the resin to remove an unbound portion of the protein, and the resultant was denatured with 6 M guanidine hydrochloride to eluate Gc protein.

### (ii) Improved Method

The purification was performed in the same manner as in the basic method except that a buffer containing 50 mM Tris-HCl (pH 7.4) and 500 mM NaC1 was used as the column buffer.

For comparing purification yield and purity between the Gc proteins purified by the respective methods, each column eluate was concentrated with an ultrafiltration membrane (Vivaspin 10,000 MWCO: GE Healthcare Limited), and the resultant was stored at -80° C., and then subjected to SDS-PAGE and dyed with Coomassie brilliant blue (CBB) R-250 (FUJIFILM Wako Pure Chemical Corporation) for visualizing the Gc protein and contaminated proteins.

A concentration of the whole protein after the purification step was determined with a Pierce(R) BCA Protein Assay Kit (Thermo Fisher Scientific). In addition, the amount and purity of the Gc protein were determined by analyzing a band concentration of each protein in the CBB dyed SDS-PAGE gel with Image J (Image Processing and Aalisis in Java).

Results of purification patterns obtained by the respective methods are illustrated in FIG. 1.

In the basic method (lane 1), Gc protein was detected as one of the principal proteins. The purify of the Gc protein was, however, as low as about 78.7%.

The improved method (lane 2) was different from the basic method, as described above, in that the salt concentration of the column buffer was increased, and that Triton X-100 and EDTA were not used. In the lane 2, contaminated proteins (a, b, and c) similar to those in the lane 1 were observed, and hence it is presumed that the purity of Gc protein is not largely affected by lack of use of Triton X-100 and EDTA.

### (Example 3) Purification of Gc Protein by Anion Exchange Chromatography

It was found that the purification purity of Gc protein was still not at a satisfactory level as described in the results below when only the purification by affinity chromatography in accordance with the method of Link et al. (Link et al., Analytical Biochemistry 157, 262-269, 1986) was performed. Therefore, the present inventors examined other purification procedures, and have found that application of anion exchange chromatography performed subsequently to the affinity chromatography is effective for improving purification purity of Gc protein.

Specific procedures are as follows:

The column eluate resulting from the improved method described above is subsequently concentrated with an ultrafiltration membrane (Vivaspin 10,000 MWCO, manufactured by GE Healthcare Limited), the resultant was dialyzed against 20 mM Tris-HCl (pH 8.0), and the thus-obtained Gc protein solution was caused to pass through RESOURCE(R) Q column (GE Healthcare Limited) having been equilibrated with 20 mM Tris-HCl (pH 8.0) to be eluted with 20 mM Tris-HCl (pH 8.0) with a stepwise concentration gradient of 0 to 1 M NaCl.

Fractions containing Gc protein were collected to be concentrated with an ultrafiltration membrane (Vivaspin 10,000 MWCO, GE Healthcare Limited), and the resultant was stored at -80° C., and then subjected to SDS-PAGE and dyed with Coomassie brilliant blue (CBB) R-250 (FUJIFILM Wako Pure Chemical Corporation) for visualizing the Gc protein and contaminated proteins.

A concentration of the whole protein after the purification step was determined with a Pierce(R) BCA Protein Assay Kit (Thermo Fisher Scientific). In addition, the amount and purity of the Gc protein were determined by analyzing a band concentration of each protein in the CBB dyed SDS-PAGE gel with Image J (Image Processing and Aalisis in Java).

Purification pattern comparison between before and after the purification by anion exchange chromatography is illustrated in FIG. 2. In a lane 1, a purification pattern obtained after the affinity purification by the improved method is illustrated, and in a lane 2, a purification pattern obtained after the secondary purification by anion exchange chromatography is illustrated.

It is thus understood that the contaminated proteins were removed and selectivity of Gc protein was increased by performing the purification by anion exchange chromatography subsequently to affinity chromatography by the improved method. The purity of Gc protein was largely improved from 78.3% to 95.0% (with substantially no contaminated proteins observed by CBB dyeing).

### Results

The purification yield and purity of Gc protein obtained after the application to the columns are all shown in the following table.

TABLE 1

YIELD AND PURITY OF GC PROTEIN PER ML OF SERUM

| | YIELD(µg) | DEGREE OF PURIFICATION(%) |
|---|---|---|
| BASIC METHOD (AFFINITY PURIFICATION) | 248 | 78.7 |
| IMPROVED METHOD (AFFINITY PURIFICATION) | 242 | 78.3 |
| IMPROVED: METHOD (ANION EXCHANGE PURIFICATION) | 147 | 95.0 |

It is understood from comparison of the affinity purification between the basic method and the improved method that the purification yield and the purity of Gc protein per mL of serum were substantially the same. This reveals that the composition change of the column buffer applied in the affinity chromatography, namely, disuse of a surfactant such as Triton X-100 and a chelating agent such as EDTA, does not largely affect the purification yield and the purity of Gc protein.

On the other hand, through the secondary purification by the anion exchange chromatography performed subsequently to the affinity purification by the improved method, although the purification yield of Gc protein was reduced from 242 µg to 147 µg per mL of serum, the purity was improved from 78.3% to 95.0%.

It was found, based on these results, that a combination of affinity chromatography and anion chromatography produces an effect advantageous to the purification purity of Gc protein. In addition, it was revealed that Gc protein can be sufficiently separated by preparing a buffer to a rather high salt concentration even when a surfactant and a chelating agent conventionally used for effectively separating a protein are not used in the column buffer used in performing the affinity chromatography.

Furthermore, according to the present disclosure, there is no need to perform a post-treatment with a hydroxyapatite column, which was performed subsequently to affinity chromatography in conventional procedures for removing a surfactant and a chelating agent.

### INDUSTRIAL APPLICABILITY

According to the present disclosure, Gc protein can be highly purified. Besides, GcMAF can be efficiently produced from Gc protein thus prepared, and hence, the present disclosure is expected to make a great contribution to research applications and clinical applications for cancer immunotherapy of extrinsic GcMAF exhibiting inhibitory effects on tumor growth and angiogenesis.

1. A method for purifying Gc protein, comprising the steps of:
(a) purification by affinity chromatography; and
(b) purification by anion exchange chromatography.

2. The method according to claim 1, wherein the step (b) of purification by anion exchange chromatography is performed after the step (a) of purification by affinity chromatography.

3. The method according to claim 1 wherein a vitamin D3-immobilized affinity column is used in the step (a) of purification by affinity chromatography.

4. The method according to claim 1, wherein a buffer having a salt concentration of more than 150 mM to 2 M is used as a buffer for washing the protein not bound to a column in the step (a) of purification by affinity chromatography.

5. The method according to claim 1, wherein a surfactant and/or a chelating agent is not used in the step (a) of purification by affinity chromatography.

6. The method according to claim 1, wherein subsequently to the step (a) of purification by affinity chromatography, a column eluate resulting from the step (a) is dialyzed against a buffer of pH 6 to 10.

7. The method according to claim 1, wherein a resin column having a cationic functional group selected from the group consisting of quaternary ammonium, diethyl aminoethyl, aminoethyl, para-aminobenzyl, and guanide ethyl is used in the step (b) of purification by anion exchange chromatography.

8. The method according to claims 1, wherein the Gc protein is eluted with concentration gradient of a salt concentration of 0 to 1 M in the step (b) of purification by anion exchange chromatography.

9. The method according to claim 1, wherein the Gc protein is purified from human plasma or human serum.

10. A method for producing a Gc protein-derived macrophage activating factor (GcMAF), comprising the steps of:
(i) purification of Gc protein according to claim 1; and subsequently
(ii) conversion to GcMAF by contacting the Gc protein with an enzyme.

11. The method according to claim 10, wherein β-galactosidase is used as the enzyme.
